(19) United States
(12) Patent Application Publication
(10) Pub. No.: US 2014/0377762 A1
(43) Pub. Date: Dec. 25, 2014

(54) METHOD FOR ENRICHING AND DETECTION OF VARIANT TARGET NUCLEIC ACIDS

(71) Applicant: 360 Genomics Ltd., Oxford (GB)

(72) Inventors: David Kelly, Oxford (GB); Antonia Cardew, Oxford (GB)

(73) Assignee: 360 Genomics Ltd., Oxford (GB)

(21) Appl. No.: 14/367,090

(22) PCT Filed: Dec. 13, 2012
(86) PCT No.: PCT/GB2012/053128

§ 371 Date: Jun. 19, 2014

Related U.S. Application Data
(60) Provisional application No. 61/577,604, filed on Dec. 19, 2011.

(51) Int. Cl. C12Q 1/68

(52) U.S. Cl. CPC C12Q 1/6806
USPC 435/6.12

(57) ABSTRACT

This invention provides methods and kits for enriching and/or detecting a nucleic acid with at least one variant nucleotide from a nucleic acid population in a sample.

### TECHNICAL FIELD

The present invention relates to the field of enrichment of one or several desired nucleic acid(s) from a population of nucleic acids in a sample, especially the enrichment of rare nucleic acids containing mutations for the purposes of detection.

### BACKGROUND TO THE INVENTION

Single nucleotide polymorphisms (SNPs) are the most common type of variation in the human genome. Point mutations are also usually SNPs but the term mutation is normally reserved for those SNPs with a frequency rarer than 1% and/or where there is a known correlative or functional association between the mutation and a disease (Gibson N J: 2006 Clin Chim Acta. 363(1-2):32-47).

There are many reasons for genotyping polymorphisms and detecting rare mutations. Rare variant detection is important for the early detection of pathological mutations, particularly in cancer. For instance, detection of cancer associated point mutations in clinical samples can improve the identification of minimal residual disease during chemotherapy and detect the appearance of tumour cells in relapsing patients. The detection of rare point mutations is also important for the assessment of exposure to environmental mutagens, to monitor endogenous DNA repair, and to study the accumulation of somatic mutations in aging individuals. Additionally, more sensitive methods to detect rare variants can revolutionise prenatal diagnosis, enabling the characterisation of foetal cells present in maternal blood.

A vast number of methods have been introduced, but no single method has been widely accepted. Many methods for detecting low-frequency variants in genomic DNA use the polymerase chain reaction (PCR) to amplify mutant and wild-type targets. The PCR products are analysed in a variety of ways, including sequencing oligonucleotide ligation restriction digestion, mass spectrometry or allele-specific hybridization to identify the variant product against a background of wild-type DNA. Other methods use allele-specific PCR to selectively amplify from the low-frequency variant, with or without additional selection. For example, by digesting PCR products with a restriction enzyme that specifically cleaves the wild-type product. Current approaches have inherent limitations due to the lack of total specificity of allele-specific primers during PCR, which creates false positives. As a result, all current approaches have limited sensitivity and accuracy (review in Jeffreys A J and May C A, 2003 Genome Res 13(10):2316-24).

Most mutation detection systems yield an assay signal that is difficult to validate in terms of the number of mutant molecules detected. This can be overcome in part by analyzing multiple samples, each containing limited DNA (typically 50 genome equivalents), to determine the number of mutant molecules in the sample. (digital PCR; Vogelstein and Kinzler 1999 Proc Natl Acad Sci U S A. 96(16);9236-41). However, the large number of PCR reactions required, combined with background noise arising from misincorporation of nucleotides during PCR is likely to limit this approach to detection levels of about 1 variant in a population of 1000 nucleic acids. Another limitation of many mutation detection procedures is that they replace the mutant site with a PCR primer sequence and yield short amplicons containing little, if any, information other than the presence of a putative mutant allele (review in Jeffreys A J and May C A, 2003).

The unifying problem behind all of these PCR approaches for detecting rare variants is replication infidelity during amplification. Jeffreys and May have provided a solution by enriching mutant DNA molecules from genomic DNA prior to analyzing'them by PCR; a process called DNA enrichment by allele-specific hybridization (DEASH) (Genome research 13:2316-2324, 2003). This method is a modification of traditional nucleic acid-enriching techniques that utilise hybridization with biotinylated DNA probes. It uses allele-specific oligonucleotides to fractionate DNA molecules differing by a single base substitution. However, this method of DNA enrichment involves multiple steps, requires large amounts of starting material and suffers from low sensitivity and efficiency.

Another enriching method is based on Restriction Fragment Length Polymorphism (RFLP), where PCR-amplified products are digested with restriction enzymes that can selectively digest either a normal or a mutated allele. Enriched PCR is a modification introduced into the RFLP analysis. The principle of this approach is to create a restriction enzyme site only within normal sequences, thus enabling selective digestion of the normal alleles amplified in a first amplification step. This prevents the non-mutant DNA from further amplification in a second amplification step while, upon subsequent amplification, the mutated alleles are enriched (U.S. Pat. No. 5,741,678; Kahn et al, 1991). This approach is limited, however, to the analysis of mutations at precise locations where restriction enzyme sites naturally occur. To overcome this limitation, one can artificially introduce restriction enzyme sites near the site of the point mutation to distinguish between normal and mutant alleles. In this approach, base-pair substitutions are introduced into the primers used for the PCR, yielding a restriction enzyme site only when the primer flanks a specific point mutation. This approach enables the selective identification of a point mutation at a known site, presumably in any gene.

Mismatched 3′ end amplification is a PCR technique which utilizes primers that have been modified at the 3′ end to match only one specific point mutation. This method relies on conditions which permit extension from primers with 3′ ends complementary to specific mismatches, whereas wild-type sequences are not extended. This procedure requires specific primers for each mutation and the PCR conditions are quite rigorous.

Recently, enrichment methods called PNA (or LNA) clamp PCR have been developed. High affinity nucleic acid analogues such as peptide-nucleic acids (PNAs) are used to inhibit nucleic acid amplification (U.S. Pat No. 5,891,825). These methods can be problematic, however. It is difficult to find the optimal conditions for the PNA/LNA clamp;

lengthy testing and redesigning are often required, and the purchase of specialised instruments may be needed. Furthermore, PNAs are expensive and difficult to synthesise and the efficiency of inhibition is often low.

EP1061135 relates to methods for detecting and identifying sequence variations in a nucleic acid sequence of interest using a detector primer. The publication concerns utilisation of diagnostics mismatches between the detector primer and the target where it occurs. The detector primer hybridizes to the sequence of interest and is extended with polymerase. The efficiency of detector primer extension is generally directly detected as an indication of the presence and/or identity of the sequence variation in the target.

WO2008/104794 describes a method for enriching a target nucleic acid with at least one variant nucleotide from a nucleic acid population in a sample. The method comprises the use of one or more enriching primers which bind with the 3′ terminus at or very close to a suspected variant. in various preferred embodiments the extension of the enriching primer serves to inhibit exponential amplification of the wild-type sequence, permitting enrichment of the rare variant.

Nevertheless, it will therefore be appreciated that the provision of novel methods and probes adapted for sensitive enrichment and detection of rare point mutations, particularly where there are multiple mutations within a particular relatively short region, would be a contribution to the art.

### SUMMARY OF THE INVENTION

The methods of the present invention allow for rapid, sensitive, and improved enrichment and optionally detection of desired nucleic adds from a nucleic acid population.

Generally speaking, in one aspect, the invention provides a method for enriching variant target nucleic adds from a population of reference nucleic acids,
wherein the nucleic acid includes a diagnostic region encompassing at least two potential variant nucleotides as compared to the reference sequence,
said method comprising:
   (a) providing the population of nucleic acids in denatured form form as first and second single. strands,
   (b) providing forward and reverse enriching primers and forward and reverse amplification primers
wherein the forward and reverse enriching primers each include a 3′ diagnostic region binding portion (DRBP) which is substantially complementary to the reference sequence of the entire diagnostic region of the first and second strands of the target nucleic acid respectively.
and wherein the nucleotide sequence of each of the forward and reverse amplification primer is substantially complementary to first and second strands of the target nucleic acid respectively at a region which is upstream of the forward and reverse enriching primer respectively,
   (c) treating the denatured nucleic acid of (a) with the primers of (b) under hybridising conditions such as to form a mixture of duplexes
wherein each duplex comprises an enriching pruner and an amplification primer annealed to each strand of the target nucleic acid,
wherein the DRBP of the enriching primer is annealed along the diagnostic region,
and wherein the amplification primer is annealed to the target nucleic acid such that the 3′ end of the amplification primer is upstream of the 5′ end of the enriching primer;
   (d) maintaining the mixture of step (c) under extension conditions, which comprise appropriate nucleoside triphosphates and a nucleic acid polymerase which is optionally thermostable, to extend the annealed primers, if extendable, to synthesize a population of nucleic acids including primer extension products.
wherein the extension of the enriching primer is inhibited by base-mismatching between the DRBP and the diagnostic region when the diagnostic region contains any one or more of the variant nucleotides, thereby allowing extension of the upstream amplification primer to pass through the diagnostic region containing any or more of the variant nucleotides,
wherein the extension of the enriching primer is promoted where the DRBP is annealed to the reference diagnostic region, thereby synthesizing an enriching primer extension product which suppresses extension initiated from the upstream amplification primer,
thereby permitting preferential exponential amplification of the nucleic acid strands which include a diagnostic region in which any one or more of the variant nucleotides are present,
   (a) repeating steps (c) and (d).

As with the methodology in WO2008/104794, in the present invention extension of the amplification primer (and hence exponential amplification) depends on extension from the enriching primer being inhibited by the presence of mutation. The unextended enriching primer is thus dissociatable from the target sequence under the extension conditions, thereby allowing extension of the amplification primer to pass through the diagnostic region containing suspected variant nucleotide and for exponential amplification to occur.

It is a key feature of WO2008/104794 that the enriching primers bind with their 3′ terminus at or very close to a suspected variant.

In the present invention multiple mutations across a larger region are detectable by the use of ‘overlapping’ enriching primers which each bind across that region on their respective strand. This means that, unlike in WO2008/104794, some or all of the variants will not be around the 3′ terminal.

Nevertheless, surprisingly, the methodology still permits highly effective and consistent enrichment of any of the mutated target present. More specifically, as shown in Example 1 and FIG. 1, the invention yields an unexpected degree of consistency of amplification across variant nucleotides placed at varying distances from the 3′ terminus of the enriching primers. When a variant nucleotide is present further from the 3′ terminus of the enriching primer it would have been expected that the efficiency of amplification would decrease, as the enriching primer would be more likely to be extended despite the presence of the variant template. However, the results herein show that variant bases placed at all positions across several nucleotides can be are amplified equally well, with CT values differing by only a few cycles.

Without wishing to be bound by theory it is believed in retrospect that the use of primers which overlap in this way permits one enriching primer to ‘compensate’ for any reduced efficiency of the other resulting from the distance between its extendible terminus and the mismatched base or bases i.e. when a variant nucleotide is placed increasingly further from the end of one enriching primer, it is at the same time placed increasingly closer to the end of the enriching primer bound to the other template strand.

Thus in preferred aspects, the method is employed to enrich a plurality of different variants occurring at different positions (e.g. 2, 3, 4, 5, or 6 or more positions) of a reference nucleic acid, where those variants occur in a diagnostic region of around 3 to 6 nucleotides in length. The enrichment is relatively consistent between the variants e.g. with CT values different only by a few cycles, and significantly differing from the CT value for the wild-type under the same conditions.

Preferably the extension product of the enriching primer itself is rendered unsuitable for exponential amplification, e.g. by making the enriching primer unsuitable for copying either by the incorporation of an appropriate moiety, or due to its sequence (which may fold on itself to prevent primer annealing).

Also provided by the present invention are related methods and materials (e.g. kits am probes).

Some particular elements of the invention will now be discussed in more detail. All combinations of the various embodiments and claims (including dependent claims) described below apply mutatis mutandis to the aspects of the invention as described herein.

### Diagnostic Region and Reference Nucleic Acid

The method of the invention permits the enrichment of variant target nucleic acids from a population of reference nucleic acids.

“Reference” as used herein will typically be the ‘normal’ sequence present in highest concentration in the sample, which in turn will typically be the ‘wild-type’ sequence. However the present invention is in principle applicable to enrichment of any sequence, particularly rare sequence, with respect to any reference sequence.

The term “diagnostic region” as used herein means that region of the target nucleic acid sequence which contains the potential variant nucleotides, Typically one of these will be a terminal nucleotide (mark the 5′ end of the region) while others will be internal nucleotides, which could be more than 3 nucleotides from the 5′ end of he region. One of the variants may mark the 3′ end of the region.

It should be appreciated that whilst the method of the present invention is of particular interest in enriching and detecting the diagnostic region of target nucleic acids containing point mutations, the method is equally applicable to enriching and detecting a diagnostic region with deletions and insertions, including deletions and insertions of more than one nucleotide. The present invention is also valuable for enriching target regions containing substitutions of more than one nucleotide. in this regard it is simply necessary to know the relevant nucleotides so that the necessary enriching primer(s) may be designed appropriately. Thus “variant nucleotide” as used herein will be understood not just to be a substitution, but also potentially several substitutions or an insertion or deletion, with the required complementarity (e.g. of probe or primer) being adjusted accordingly.

### Enriching Primer

The term“enriching primer” as used herein to refer to the primer that has a nucleotide sequence such that it is substantially complementary to a diagnostic region where the suspected variant nucleotides are located. When an enriching primer anneals to a target sequence, it may be extended or may not be extended depending on the presence or absence of the suspected variant nucleotides. More specifically, when it anneals to the diagnostic region containing the suspected variant nucleotides, the annealed enriching primer is non-extendable, or at least extension is significantly inhibited, whereby the enriching primer is dissociated from the target sequence under the extension conditions, thereby allowing extension of the amplification primer to pass through the diagnostic region containing suspected variant nucleotides. In various embodiments described herein this dissociation may preferably be achieved by the use of temperature control (i.e. the extension conditions include or are followed by a melting condition which is capable of removing un-extended enriching primer). In others it may be achieved by use of an exonuclease activity. When it anneals to the diagnostic region containing the corresponding normal nucleotides, the annealed enriching primer is extended to synthesize the enriching primer extension product whereby the extension from an upstream amplification primer is blocked by the enriching primer extension product.

### Extension of the Enriching Primer when Annealed to the Reference Diagnostic Region

Template-dependent extension of the oligonucleotide primer(s) is catalyzed by a polymerizing agent in the presence of adequate amounts of the four deoxyribonucleoside triphosphates (dATP, dGTP, dCTP, and dTTP), or analogues of these as discussed above. in a reaction medium comprised of the appropriate salts, metal cations and pH buffering system. Suitable polymerizing agents are enzymes known to catalyze primer- and template-dependent DNA synthesis. The reaction conditions for catalyzing DNA synthesis with these DNA polymerases are well known in the art.

As noted above, the diagnostic region binding portion (DRBP) of the enriching primer is “substantially complementary” to the entire diagnostic region of the reference sequence “Substantially complementary” means that the primers must be sufficiently complementary to hybridize with their respective strands. Therefore, the primer sequence need not reflect exact sequence of the template. However it must be sufficiently complementary to prime extension when bound to the reference sequence. For this reason typically the t to DRBP will be entirely complementary to the diagnostic region. However it will be appreciated that even where the enriching primer comprises a 3′ DRBP which is complementary to the reference sequence of the entire diagnostic region a non-complementary nucleotide fragment may be attached to the 5′-end of the primer, with the remainder of the primer sequence being complementary to the diagnostic portion of the target base sequence.

### Blocking of Extension of Amplification Primer by Enriching Primer Extension Product

When an enriching primer is extended, an upstream amplification primer or an upstream enriching primer for another diagnostic region is also extended but will stop when their extension strands reach the downstream enriching primer extension product.

In the practice of the invention, the enriching primer must first be annealed to the diagnostic region (and, under appropriate circumstances, extended) before the amplification primer extension reaches and/or blocks the enriching primer binding site. To achieve this, a variety of techniques may be employed. One can position the enriching primer so that the 5′ end of the enriching primer is relatively far from the 3′ end of the amplification primer, thereby giving the enriching primer more time to anneal. One can also use an enriching primer with a higher Tm than the amplification primer. For example, the enriching primer can be designed to be longer than the amplification primer. The nucleotide composition of the enriching primer can be chosen to have greater G/C content and, consequently, greater thermal stability than the amplification primer. In a similar fashion, one can incorporate into the enriching primer modified nucleotides which contain base analogues that form more stable base pairs than the bases that are typically present in naturally occurring nucleic acids.

The thermocycling parameters can also be varied to take advantage of the differential thermal stability between the enriching primer and amplification primers. For example, following the denaturation step in thermocycling, an intermediate temperature may be introduced which is permissible for enriching primer binding but not for amplification primer binding; the temperature is then increased to the extension temperature (for example 72° C.), whereby permitting extension of the matched enriching primer and melting away of unextended enriching primer. The cycles of an intermediate temperature and extension temperature can be repeated as many times as desirable to allow the matched enriching primer to extend on as many target templates as possible. The temperature can then be reduced to permit amplification primer annealing and extension.

When the amplification primer is annealed to the target nucleic acid such that the 3′ end of the amplification primer is upstream of the 5′ end of the enriching primer it will typically be close enough to the 5′ end of the enriching primer to ensure that both can bind to a given sample of template DNA, but far enough away to ensure that the enriching primer has a chance to be extended without first being displaced.

Thus in some embodiments the gap between the two may for example be more than, less than or equal to 20, 21, 22, 23, 24, 25, 26, 27, 28, 29 or 30 or more nucleotides, Preferably the gap is in the range of 20 to 150 nucleotides e.g. 20 to 90, 20 to 80 nucleotides.

### Prevention of Amplification of Enriching Primer Extension Product

Since an enriching primer extension product is produced in the presence of the reference sequence, it is preferred that the enriching primer be adapted such that replication of all or part of said enriching primer is blocked.

The enriching primer will typically comprise a moiety that renders the extension product of the enriching primer unsuitable for an exponential amplification. In one embodiment, the moiety may be a blocking moiety (or referred to as a non-copiable moiety), wherein the replication of all or part of said enriching-primer is blocked, whereby the primer extension molecule generated from a template of the enriching primer extension strand is not suitable as a template for a further primer extension as it lacks a primer binding site.

In principle, the non-copiable moiety (blocking moiety) included in the enriching primer may be any entity which is not recognized as a suitable template by a polymerase. It is desirable that the blocking moiety (for example dR-biotin, dR-amine) is capable of insertion in synthetic oligonucleotides by incorporation of appropriate precursors (e.g. phosphoramidites) during in vitro synthesis of the oligonucleotide

Thus the blocking moiety may be hydrocarbon arm, an HEG, non-nucleotide linkage, abasic ribose, nucleotide derivatives or a dye. The blocking moiety may be located at less than 18 nucleotides away from 3′ terminus of the enriching primer. it is preferred that the blocking moiety may be located at less than 6 nucleotides away from 3′ terminus of the enriching-primer. it is more preferred that the blocking moiety may be located at less than 3 nucleotides away from 3′ terminus of the enriching primer.

In another embodiment the enriching primer may comprise additional sequences 5′ of the priming portion (DRBP) that may or may not be complementary to a target sequence, this additional sequence may be referred to as a tail. The tail may be utilised to facilitate detection of extension products or inhibit copying of the primer, as described in WO2008/104794 the disclosure of which is herein incorporated by reference. Briefly, in one embodiment, the enriching primer comprises a 5′ tail sequence which is complementary or substantially complementary to a primer binding site on the enriching primer extension product. The enriching primer extension product, upon being subjected to denaturing and hybridising conditions, folds back to form a stem-loop structure which prevents a further primer binding.

In a different embodiment the enriching primer may be an ordinary oligonucleotide primer made of natural nucleotides and a phosphodiester linkage—in other words, it might not comprise a non-nucleotide, a linkage chemical moiety or modified nucleotides. In this embodiment, a part or whole of the enriching primer is degradable by a nuclease activity, thereby allowing the amplifying primer to pass straight through. When the enriching primer anneals to the diagnostic region containing the corresponding normal nucleotides on the target sequence, the enriching primer is extended by a DNA polymerase with a 5′ exonuclease activity under an extension condition which comprises at least one modified deoxynucleoside triphosphate. A part or whole of the enriching primer is degraded by said 5′ exonuclease activity, whereas a part or whole of the extended strand is resistant to cleavage. This extended, resistant, strand can thus ‘block’ the amplification primer.

In summary, if the enriching primer comprises a non-copiable (blocking) moiety or the part of the enriching primer on the enriching primer extension product is degraded, a further amplification of the enriching primer extension product is prevented. Herein the expression “further amplification” means specifically an exponential amplification. In some embodiments, a linear (or polynomial amplification) replication of the enriching primer extension product, although not the whole product, is allowed. In another embodiment, both linear and exponential replication of the enriching primer extension product is prevented. When an annealed enriching primer cannot be extended, it will be dissociated from the template, allowing an exponential amplification of the target sequence flanked by two amplification primers, thereby enriching the desired target sequence.

### Inhibition of Extension of the Enriching Primer when Annealed to a Variant Diagnostic Region

When the enriching primer anneals to the diagnostic region containing the variant nucleotide(s), the mismatched 3′ DRBP of the enriching primer cannot be efficiently extended and the enriching primer will be dissociated from the template under the extension conditions. Thus amplification of the diagnostic region containing the variant nucleotide(s) by the amplification primers can proceed.

Since the diagnostic region binding portion (DRBP) of the enriching primer is “substantially complementary” (typically exactly complementary) to the reference sequence, it will contain one or more mismatches with respect to a diagnostic region.

It is preferred that a 3′ terminal nucleotide of the enriching primer is selected to base pair with the normal nucleotide at one of the variant positions. However, typically in the practice of the present invention at least one of the variant positions will be greater than 3 nucleotides from the 3′ terminus of one of the enriching primers. However, as explained above, surprisingly the use of twin, overlapping primers means that the exponential amplification of the variant containing sequence is nevertheless inhibited.

Those skilled in the art will appreciate that appropriate conditions should be adopted to ensure that synthesis of a primer extension product does not occur, or is greatly inhibited, when the 3′ DRBP binds to the non-complementary ‘variant’ sequence. Artefactual results could in principle arise from an annealing/incubation temperature that is too low (in which case the temperature may be increased), too long of an incubation/annealing time (in which case the time may be reduced), a salt concentration that is too high (in which case the salt concentration may be reduced), an enzyme or nucleoside triphosphate concentration that is too high, an incorrect pH, or an incorrect length of oligonucleotide primer. Artefactual results may be avoided by deliberately introducing one or more further mismatched residues, or if desired, deletions or insertions, within the diagnostic primer to destabilise the primer by further reducing the binding during hybridisation. In the light of the disclosure herein such would not present an undue burden to those skilled in the art.

### Polymerases

The disclosed methods make use of nucleic acid polymerase for primer extension. Any nucleic acid polymerase can be used.

Preferably, since it is intended that the extension of the enriching primer blocks the extension initiated from the amplification primer, the polymerases used preferably do not have a strand displacement activity, such as Tag DNA polymerase or the Stoffel fragment of the Taq polymerase.

In other embodiments (described in more detail below) wherein the enriching primer comprises a 3′ blocking moiety which, if not removed, prevents primer extension, the DNA polymerase comprises a proof-reading activity or pyrophosphorolysis activity, such as Pfu, PWO, Pfx, Vent DNA polymerases, AmpliTaqFS or ThermoSequenase.

It is particularly preferred that the DNA polymerase is a thermostable DNA polymerase.

### Denaturation Conditions

In the method described herein, a sample is provided which is suspected to contain the target nucleic acid and the nucleotide variant(s) of interest. The target nucleic acid contained in the sample may originally be double-stranded genomic DNA or cDNA which is then denatured to first and second complementary single strands, using any suitable denaturing method including physical, chemical, or enzymatic means that are known to those of skill in the art. A preferred physical means for strand separation involves heating the nucleic acid until it is completely (>99%) denatured. Typical heat denaturation involves temperatures ranging from about 80° C. to about 105° C., for times ranging from a few seconds to minutes.

As an alternative to denaturation, the target nucleic acid may initially exist in a single-stranded form in the sample, such as single-stranded RNA or DNA viruses.

### Hybridisation Conditions

The denatured nucleic acid strands are then incubated with oligonucleotide primers under “hybridisation conditions”; conditions that enable the binding of the primers to the single nucleic acid strands.

These conditions will typically be in the range of 15 seconds to 1 minute, more usually around 20 to 30 seconds, at a temperature of around 40 to 60° C. e.g. 20 seconds at 50° C. The Tm of the primers can be used to help determine the optimal annealing temperature, using methods well known to those skilled in the art.

### Extension Conditions

The duplexes are then incubated with oligonucleotide primers under “extension conditions”. These conditions will typically be in the range of 20 seconds to 1 minute, more usually around 25 to 40 seconds, at 50 to 70° C. e.g. 30 seconds at 60° C. Again those skilled in the art will be readily able to selected extension conditions appropriate to the system they are using.

### Melting Conditions

Between the annealing and extension steps any partially annealed enriching primer can be melted off the variant template to allow full amplification by the amplifying primer Conditions used should be in the range of 1 to 5 seconds at 60 to 80° C. e.g. 1 second at 70° C. Again those skilled in the art will be readily able to selected melting conditions appropriate to the system they are using.

### Detection and Probes

The method of the present invention may further comprise detecting the enriched desired sequence. Detection may be carried out simultaneously with the process of enrichment, for example real-time detection. A detection probe may be included in an enrichment/amplification reaction. Any detection probe can be used Alternative detection may be carried out at the end of the enrichment reaction. A probe may be added at the beginning or end of said enrichment reaction. A melting curve analysis may be performed to detect the suspected variant nucleotide present in the enriched amplification product. It is preferred that a quantitative data is obtained by detection.

It is also possible that detection or verification of the enrichment of the desired nucleic acid is carried out after enrichment, which may be accomplished by a variety of methods, such as a real-time PCR or DNA sequencing.

In some embodiments the enriching primer or amplification primer may be a labeled oligonucleotide. The term “label” as used herein refers to any atom or molecule which can be used to provide a detectable (preferably quantifiable) signal, and which can be attached to a nucleic acid or protein. Labels may provide signals detectable by fluorescence, radioactivity, colorimetry, gravimetry, magnetism, enzymatic activity and the like.

Probes may be used in the present invention to assist in detection of amplification products. Probes suitable for such a purpose are well known to those skilled in the art. Briefly, the probe, normally, does not contain a sequence complementary to the sequence(s) used to prime the amplification. But in some embodiments, a probe does contain a sequence complementary to a part of a primer. Generally the 3′ terminus of the probe will be “blocked” to prohibit incorporation of the probe into a primer extension product. But in some embodiments, some probes are also working as primers and therefore are not blocked at the 3′ terminus. “Blocking” can be achieved by using non-complementary bases or by adding a chemical moiety such as biotin or a phosphate group to the 3′ hydroxyl of the last nucleotide, which may, depending upon the selected moiety, serve a dual purpose by also acting as a label for subsequent detection or capture of the nucleic acid attached to the label. Blocking can also be achieved by removing the 3′-OH or by using a nucleotide that lacks a 3′-OH such as a dideoxynucleotide.

Particular probes which can advantageously be used in conjunction with the present invention, including so-called “bridge-probes”, are described in WO2008/104794.

Modifications of the probe that may facilitate probe binding include, but are not limited to, the incorporation of positively charged or neutral phosphodiester linkages in the probe to decrease the repulsion of the polyanionic backbones of the probe and target (see Letsinger at al., 1988, J. Amer. Chem Soc. 110:4470); the incorporation of alkylated or halogenated bases, such as 5-bromouridine, in the probe to increase base stacking; the incorporation of ribonucleotides into the probe to force the probe:target duplex into an “A” structure, which has increased base stacking; and the substitution of 2,6-diaminopurine (amino adenosine) for some or all of the adenosines in the probe; the incorporation of nucleotide derivatives such as LNA (locked nucleic acid), PNA (peptide nucleic acid) or the like.

It has been known that in homogeneous hybridization assays, two interactive fiuorophores can be attached to the ends of two different oligodeoxyribonucleotide probes or to the two ends of the same oligodeoxyribonucleotide probe. A target nucleic acid reveals itself by either bringing the donor fluorophore and the acceptor fluorophore close to each other, permitting energy transfer between them to occur, or by separating them from each other, precluding the transfer of energy (Marras S. A. E. at al 2002, Nucleic Acids Res, 30(21)). The earliest formats for homogeneous hybridization assays utilized a pair of oligodeoxyribonucleotide probes labeled at their respective 5′ and 3′ ends that were designed to bind to adjacent sites on a target strand, thereby bringing a donor and acceptor moiety close to each other (Patent no. EPO070685 and Cardullo R. A. at at 1988).

A second approach utilizes a pair of mutually complementary oligodeoxyribonucleotides, in which one of the oligodeoxyribonucleotides serves as a probe for a single-stranded target sequence. The 5′ end of one oligodeoxyribonucleotide is labeled with a donor fluorophore and the 3′ end of the other oligodeoxyribonucleotide is labeled with an acceptor fluorophore, such that when the two oligodeoxyribonucleotides are annealed to each other, the two labels are close to one another. Since small complementary oligodeoxyribonucleotides bind to each other in a dynamic equilibrium, target strands compete for binding to the probe, causing the separation of the labeled oligodeoxyribonucleotides (Morrison L. E. et al 1989, Anal. Biochem., 183:, 231-244).)

In a third approach, the donor and acceptor fluorophores are attached to the ends of the same oligodeoxyribonucleotide, which serves as the probe. Since an oligodeoxyribonucleotide in solution behaves like a random coil, its ends occasionally come close to one another, resulting in a measurable change in energy transfer. However, when the probe binds to its target, the rigidity of the probe-target helix keeps the two ends of the probe apart from each other, precluding interaction between the donor and the acceptor moieties (Parkhurst K M. and Parkhurst, L. J. 1995 Biochemistry, 34:. 285-292).

In the fourth approach, single-stranded oligodeoxyribonucleotides called molecular beacons possess short additional sequences at either end of a probe sequence that are complementary to one another, enabling terminal labels to be in close proximity through the formation of a hairpin stem. Binding of this probe to its target creates a relatively rigid probe-target hybrid that causes the disruption of the hairpin stem and the removal of the donor moiety from the vicinity of the acceptor moiety, thus restoring the fluorescence of the donor (Tyagi S. and Kramer, F. R. 1996, Nat. Biotechnol., 14:, 303-308). In addition to these hybridization-based schemes and their variations, dual-labeled randomly coiled probes that bind to template strands during PCR, can be enzymatically cleaved by the 5′→3′ endonuclease activity of DNA polymerase (“TaqMan”™ probes), separating the donor and acceptor moieties and enabling nucleic acid synthesis to be monitored in real time (Heid C. A., Stevens, J., Livak, K. J. and Williams, P. M. 1996, Genome Res., 6:, 986-994).

If an acceptor fluorophore is brought closer to a donor fluorophore within the range 20-100 angstroms the fluorescence intensity of the acceptor fluorophore increases, whereas the fluorescence intensity of the donor fluorophore decreases. This is due to an increase in the efficiency of fluorescence resonance energy transfer (FRET) from the donor to the acceptor fluorophore. However, if the two moieties are brought any closer, the fluorescence intensities of both the donor and the acceptor fluorophores are reduced. At these intimate distances, most of the absorbed energy is dissipated as heat and only a small amount of energy is emitted as light, a phenomenon sometimes referred to as static or contact quenching (Lakowicz J. R. 1999, Principles of Fluorescence Spectroscopy. Kluwer Academic/Plenum Publishers. New York, N.Y.).

In adjacent probes and in randomly coiled probes, the donor and the acceptor moieties remain at such a distance from each other that FRET is the predominant mechanism of quenching. On the other hand, when competitive hybridization probes and molecular beacons are not hybridized to targets, the two moieties are very close to each other and contact quenching is the predominant mechanism of quenching. One of the useful features of contact quenching is that all fluorophores are quenched similarly, regardless of whether the emission spectrum of the fluorophore overlaps the absorption spectrum of the quencher, one of the key conditions that determines the efficiency of FRET (Tyagi S., Bratu, D. P. and Kramer, F. R. 1998, Nat Biotechnol 16:, 49-53).

A further simplification of homogeneous assays that utilize fluoresceritly labeled probes is the use of non-fluorescent dyes as acceptors or quenchers. Quenching by non-fluorescent dyes enables changes in the intensity of fluorescence to be measured directly, rather than as an alteration in the shape of the emission spectrum, which is more difficult to monitor. This improvement has also led to a higher degree of multiplexing, as the part of the spectrum that would have been occupied by the fluorescence of the quencher can instead be reserved for the fluorescence of additional fluorophores for the detection of more targets (Marras S. A., Kramer, F. R. and Tyagi, S. 1999, Genet. Anal., 14:, 151-156).

Recently, a number of unique non-fluorescent quenchers ranging from nucleotides to gold particles, have been introduced for use in fluorogenic probes (Dubertret B., Calame, M. and Libchaber, A. J. 2001, Nat. Biotechnol., 19:, 365-370). Quenching efficiencies up to several thousand-fold have been reported for some of these quenchers.

In the prior art discussed above, researchers have mainly used approaches that depend on detecting increased fluorescence of the labels upon hybridisation of probe to target sequence. FRET is the main mechanism behind these approaches. The hybridization probes (U.S. Pat. No. 6,174,670) uses two fluorescent dyes which are dependent on FRET effect. This approach limits the number of multiple targets which can be detected in a single reaction.

It is preferred that for effective contact quenching, the fluorophores and quencher are at a distance of about 0-10 nucleotides, It is more preferred that the fluorophores and quencher are at a distance of about 0-5 nucleotides. It is most preferred that the fluorophores and quencher are at a distance of about 0-2 nucleotides. The quencher is preferably a non-fluorescent entity. The quencher may be a nanoparticle. A nanoparticle may be a gold nanoparticle. It is also possible that the quencher is a G residue or multiple G residues.

In the above embodiments, the>labels may be interactive fluorophores or non-fluorophore dyes or any entity. One example of such interactive labels is a fluorophore-quencher pair. “Fluorophore” as used herein to refer to moieties that absorb light energy at a defined excitation wavelength and emit light energy at a different defined wavelength. Examples of fluorescence labels include, but are not limited to: Alexa Fluor dyes, Cascade Blue, Cascade Yellow, Cyanine dyes, FAM, PyMPO, Pyrene and Texas Red. As used herein, the term “quencher” includes any moiety that is capable of absorbing the energy of an excited fluorescent label when it is located in close proximity to the fluorescent label and capable of dissipating that energy. A quencher can be a fluorescent quencher or a non-fluorescent quencher, which is also referred to as a dark quencher. The fluorophores listed above can play a quencher role if brought into proximity to another fluorophore, wherein either FRET quenching or contact quenching can occur. It is preferred that a dark quencher which does not emit any visible light is used. Examples of dark quenchers include, but are not limited to, DABCYL, diarylrhodamine carboxylic acid, nucleotide analogs, nucleotide G residues, nanoparticles, and gold particles.

Fluorescent dyes such as SYBR green can be included in the reaction mix and used to produce amplification plots and melt curves during the reaction. If amplification takes place at around 20 PCR cycles this indicates that variant template was present in the starting material and has been amplified. This method can be used to determine which samples to obtain sequence data for. There are several methods which can be used to obtain sequencing information. Preferred methods are homogenous (they do not require the reaction vessel to be opened between PCR and detection): such as incorporating a HyBeacons probe into the reaction. HyBeacons probes anneal to the region of interest and fluoresce, providing information about the sequence under the probe. Other, non-homogenous methods (requiring addition of further reagents after PCR) include but are not limited to sequencing, multiplex probing and microarrays. Sequencing is the most preferred outcome after the use of a homogenous assay-probe system. A range of sequencing methodologies are available including but not limited to sanger sequencing (performed using dideoxynucleotides attached to fluorophores or radioactive moieties) and pyrosequencing (each nucleotide is added in turn and the product of extension is measured).

Multiplex probing can also be used for detection of a variety of mutations in one reaction, generally by adding a probe with a different fluorophore to detect each sequence. Microarrays or ‘gene chips’ are another method which can be used to obtain sequencing information after the assay has been carried out. The arrays or ‘chips’ each contain thousands of DNA probes which bind to specific DNA sequences in the sample and report their presence.

### Applications

The improved methodology allows for rapid and sensitive detection of genetic variations, for example in nucleic acids in samples from patients with genetic diseases or neoplasias, for use in assays to detect and monitor gene expression or ‘rare’ mutations in a test sample.

Thus it should be appreciated that the use of the terms “Variant nucleotide”, “normal nucleotide” or “mutant nucleotide” is situation dependent and may be interchangeable. In one situation, a nucleotide may be called variant or mutant, but in another situation, it may be called ‘normal’ nucleotide (e.g. because it was obtained from an unusual source where it was present in greatest concentration in the sample).

Thus the methodology has application in a number of fields including but not limited to oncology, forensics, infectious disease agent genotyping, early detection of resistance conferring mutations and foetal diagnostics. In oncology the technology has many uses including but not limited to detection of cancer causing point mutations in clinical samples. In forensics the technology has application in enriching for particular SNPs from a sample of mixed DNA. The technology could also be used for infectious disease agent genotyping, such as HIV or influenza, for the early detection of resistance-conferring mutations. Similarly the method can he used for early detection of drug resistant mutations in bacteria and organ transplant therapy. Non invasive foetal DNA testing, which requires low sensitivity, could also be carried out using this technology.

A preferred application is the detection of variant nucleotides, which may be somatic mutations such as those found in the KRAS gene (see Kranenburg “The KRAS oncogene: past, present, and future.” Biochim Biophys Acta. 2005 Nov. 25; 1756(2):81-2. Epub 2005 Oct. 25.

Thus in one embodiment of the invention the variant target nucleic acids are variants of the KRAS gene (see http://www.genecards.org/cgi-bincarddisp.pl?gene=KRAS; also Example 1 below). The diagnostic region may thus encompass codons 12 and 13 of the KRAS sequence. More specifically the method may be used to enrich variant comprising one or more of the following mutations (three possible substitutions at one or more of first two bases in each codon (i.e. 4×3=12 possible substitutions, equivalent to 256 different possible sequences).

| | |
|---|---|
| Codon 12: | Codon 13: |
| Gly 12 Asp GGT > GAT | Gly 13 Asp GGC > GAC |
| Gly 12 Ala GGT > GCT | Gly 13 Ser GGC > AGC |
| Gly 12 Val GGT > GTT | Gly 13 Arg GGC > CGC |
| Gly 12 Ser GGT > AGT | Gly 13 Val GGC > GTC |
| Gly 12 Arg GGT > CGT | Gly 13 Cys GGC > TGC |
| Gly 12 Cys GGT > TGT | Gly 13 Ala GGC > GCC |

As described above, the method may be used to consistently enrich mutation-containing KRAS sequences against a background of ‘normal’ (reference) sequence.

Non-limiting example primers suitable for use in this embodiment are described in Example 1.

In another preferred embodiment, the variant target nucleic acids are variants of the EGFR gene (http://www.genecards.org/cgi-bin/carddisp.pl?gene=EGFR).

### Kits

Reagents employed in the methods of the invention can be packaged into assay kits. Assay kits include enriching primers for each diagnostic region of a target nucleic acid sequence, the 3′ DRBP of an enriching primer being complementary to the normal diagnostic region, such that, when in use, an extension product of the enriching primer is synthesized when said terminal nucleotide of the enriching primer anneals to the diagnostic region with the corresponding normal nucleotide, whereas the enriching primer is not extendable when said terminal nucleotide of the enriching anneals to the diagnostic region containing the variant nucleotide; and corresponding first and second primers for amplifying a target sequence containing the diagnostic region to which the enriching primer anneals.

The kit may also contain other suitably packaged reagents and materials needed for amplification, for example amplification primers, buffers, dNTPs and/or polymerizing means, and detection analysis, as well as instructions for conducting the assay.

In another embodiment, an assay kit includes probes and primers for each diagnostic region of a target nucleic acid sequence, wherein the probes and primers comprise labels which are contact quenching pairs and upon hybridisation to target nucleic acid the labels are in a contact quenching relationship. The first region of the template nucleic acid may be a region of interest on a target nucleic acid, which can be a diagnostic region with suspected variant nucleotides.

Thus certain kits of the invention are those adapted for performance of the methods defined herein—for example including combinations of enriching and amplification primers and written instructions for performing any of the methods defined herein. The kit may also include a nucleic acid polymerase e.g. comprising a 5′ exonuclease activity.

### Definitions and Preferred Embodiments

The target nucleic acid may be in a “Sample”. A sample refers to any substance containing or presumed to contain nucleic acid and includes a sample of tissue or fluid isolated from an individual or individuals.

As used herein, the terms “nucleic acid”, “polynucleotide” and “oligonucleotide” refer to primers, probes, oligomer fragments to be detected, oligomer controls and unlabeled blocking oligomers and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule.

Primers herein are typically comprised of a sequence of approximately at least 6 nucleotides, preferably at least 10-12 nucleotides, and more preferably at least 15-20 nucleotides corresponding to a region of the designated nucleotide sequence.

In principle the “target” may be single stranded in the sample. ever as described herein the target would then need to be copied in the course of, or prior to, the reaction, as double stranded nucleic acid which is denatured to permit annealing of primers to both strands.

As used herein, the term “target sequence” or “target nucleic acid sequence” refers to a region which is to be amplified and optionally detected. The target sequence, which is the object of amplification and detection, can be any nucleic acid. The target sequence can be RNA, cDNA, genomic DNA or DNA from a disease-causing microorganism or virus. The target sequence can also be DNA treated by chemical reagents, various enzymes and physical exposure. A target nucleic acid sequence of interest in a sample may appear as single-stranded DNA or RNA such as cDNA, mRNA, other RNA or as separated complementary strands. Separating complementary strands of target nucleic acid may be accomplished by physical, chemical or enzymatic means.

The term “primer” as used herein refers to an oligonucleotide, whether occurring naturally or produced synthetically, which is capable of acting as a point of initiation of synthesis when placed under conditions in which synthesis of a primer extension product complementary to a nucleic acid strand is induced i.e., in the presence of nucleotides and an agent for polymerization such as DNA polymerase and at a suitable temperature and buffering conditions. The primer must be sufficiently long to prime the synthesis of extension products in the presence of the inducing agent. The exact lengths of the primers will depend on many factors, including temperature, source of primer and use of the method.

The term “complementary to” is used herein in relation to nucleotides to mean a nucleotide that will base pair with another specific nucleotide. Thus adenosine triphosphate is complementary to uridine triphosphate or thymidine triphosphate and guanosine triphosphate is complementary to cytidine triphosphate. It is appreciated that whilst thymidine triphosphate and guanosine triphosphate may base pair under certain circumstances they are not regarded as complementary for the purposes of this specification. It will also be appreciated that whilst cytosine triphosphate and adenosine triphosphate may base pair under certain circumstances they are not regarded as complementary for the purposes of this specification. The same applies to cytosine triphosphate and uracil triphosphate.

As used herein it will be understood that “duplex” refers to double stranded nucleic acid (formed between the primer or primers, and the target nucleic acid having the variant or normal nucleotides) and does not imply that only a single enriching primer is bound—indeed typically both an enriching primer and amplification primer will be present in the duplex with the target.

The term “amplification primer” is used herein to refer to a primer that is capable of hybridising to the target sequence upstream of the enriching primer and is used for amplification. When there are first and second “amplification primers” used in a reaction, the pair of amplification primers amplify a target region spanning (extending beyond) the diagnostic region. One “amplification primer” has a nucleotide sequence such that it is capable of hybridising to an extension product of the other amplification primer, after separation from its complement, whereby one primer extension product serves as a template for synthesis of an extension product of another amplification primer.

When two different, non-overlapping oligonucleotides anneal to different regions of the same linear complementary nucleic acid sequence, and the 3′ end of one oligonucleotide points toward the 5′ end of the other, the former may called the “upstream” oligonucleotide and the latter the “downstream” oligonucleotide.

As used herein, the term “probe” refers to a labeled oligonucleotide that forms a duplex structure with a sequence in the template nucleic acid, due to complementarity of at least one sequence in the probe with a sequence in the template region.

The term “nucleoside triphosphate” is used herein to refer to nucleosides present in either DNA or RNA and thus includes nucleosides which incorporate adenine, cytosine, guanine, thymine and uracil as base, the sugar moiety being deoxyribose or ribose. In general deoxyribonucleosides will be employed in combination with a DNA polymerase. It will be appreciated however that other modified bases capable of base pairing with one of the conventional bases adenine, cytosine, guanine, thymine and uracil may be employed. Such modified bases include for example 8-azaguanine and hypoxanthine.

The term “nucleotide” as used herein can refer to nucleotides present in either DNA or RNA and thus includes nucleotides which incorporate adenine, cytosine, guanine, thymine and uracil as base, the sugar moiety being deoxyribose or ribose. It will be appreciated, however, that other modified bases capable of base pairing with one of the conventional bases, adenine, cytosine, guanine, thymine and uracil, may be used in the primers employed in the present invention. Such modified bases include for example 8-azaguanine and hypoxanthine.

In one embodiment of the invention, wherein a DNA polymerase with 5′ exonuclease activity is used, the extension condition comprises all four deoxynucleoside triphosphates, at least one of which is substituted (or modified). The substituted deoxynucleoside triphosphate should be modified such that it will inhibit cleavage by the 5′ exonuclease of the DNA polymerase. Examples of such modified deoxynucleoside triphosphates can include 2′-deoxyadenosine 5′-O-(1-thiotriphosphate), 5-methyldeoxycytidine 5′-triphosphate, 2′-deoxyuridine 5′-triphosphate and 7deaza-2′-deoxyguanosine 5°-triphosphate.

### Particular Embodiments

Reference herein to the “appropriate terminal nucleotide” means the terminal nucleotide or nucleotides of the primer from which, in use, synthesis would be initiated if possible. Since in general the agent for polymerisation would initiate synthesis at the 3′ end of the primer, the appropriate terminal nucleotide would in general be close to the 3′ terminus of the enriching primer. To prevent the 3′ terminal nucleotide or other nucleotides of the enriching primer being digested by a nuclease activity, the enriching primer may comprise modified nucleotides or linkages which render the whole or part of the enriching primer resistant to nuclease cleavage. It is preferred that the last 5 nucleotides or linkages at the 3 end and/or 5′ end are modified such that the enriching primer is resistant to nuclease cleavage. It is more preferred that the last nucleotide or linkage at 3′ end and/or 5′ end is modified such that the enriching primer is resistant to nuclease cleavage. Any type of modification which renders the primer resistant to exonuclease cleavage can be used. Examples include phosphorothioate linkage, methylphosphonate linkage, LNA, PNA, Oligo-2′-OMe-nucleotides or the like.

in some embodiments of the present invention, a nucleic acid template capable of forming a stem-loop structure may be created by an extension of a first amplification primer with a 5′ tail sequence. The 5′ tail sequence comprises nucleotide or non-nucleotide sequence complementary to the binding site of the second amplification primer, in other words, the 5′ tail sequence comprises nucleotide or non-nucleotide sequence identical or substantially identical to the sequence of the second primer. The first and second amplification primers are capable of hybridising to the extension product of the second and first amplification primers, respectively. Alternatively, the nucleic acid template capable of forming a stem loop structure may be created by an extension of first and second amplification primers with the same 5′ tail sequence. The 5′ tail sequence comprises nucleotide or non-nucleotide sequence complementary to the binding site of a third amplification primer. In other words, the 5′ tail sequence comprises nucleotide or non-nucleotide sequence identical or substantially identical to the sequence of the third primer. The third primer may be an arbitrary universal primer unrelated to the target sequence.

As explained above, the enriching primer anneals to the diagnostic region and may or may not be efficiently extended depending on whether or not variations are absent. The amplification primer anneals to the nucleic acid strand upstream of the enriching primer and, when extended, passes through the diagnostic region or its extension is blocked by the enriching primer extension product. The reaction includes a first amplification primer and a first enriching primer annealing to the first strand of the target sequence, and a second amplification primer and a second enriching primer annealing to the second strand of the target sequence, which is complementary to the first strand of the target sequence.

In one embodiment, the nucleic acid template forms a stem-loop structure. The nucleic acid template capable of forming a stem-loop structure may be created by an extension of a first amplification primer on the target nucleic acid in the sample. The first amplification primer comprises a 5′ tail sequence, which comprises nucleotide or non-nucleotide sequence complementary to the binding site of second amplification primer The first and second amplification primers are capable of hybridising to the extension product of the second and first amplification primers, respectively, after separation from its complement or from the stem-loop structure. The nucleic acid template may be created by extensions of both first and second amplification primers on the target nucleic acid in the sample. In another embodiment, both first and second amplification primers comprise the same 5′ tail sequence, wherein said 5′ tail sequence comprises nucleotide or non-nucleotide sequence complementary to the binding site of a third amplification primer. The third amplification primer is present at concentrations that greatly exceed the concentrations of the first and second amplification primers in the reaction. The third amplification primer may be present in the reaction at a concentration of least 2 times more than the concentration of the first and second amplification primers. Preferably, the third amplification primer may be present in the reaction at a concentration of at least 3 times more than the concentration of the first and second amplification primers.

The amplification primers are selected so that their relative positions along a duplex sequence are such that an extension product synthesized from the first amplification primer, when the extension product is separated from its template (complement), serves as a template for the extension of the second amplification primer.

The invention will now be further described with reference to the following non-limiting examples. Other embodiments of the invention will occur to those skilled in the art in light of these.

The disclosure of all references cited herein, inasmuch as it may be used by those skilled in the art to carry out the invention. is hereby specifically incorporated herein by cross-reference.

### FIGURES

FIG. 1—KRAS 12113 Global Assay Using Mismatch Enriching Primers

Mis-match is generated on both forward and reverse enriching primers, enforcing a block to exponential amplification in both directions. Although the block from any one primer is weakened as the mis-match moves further from the 3′ terminus, the compensating move towards the terminus of the opposite primer can balance out the overall blocking capacity of the assay.

FIG. 2—Suppression of Amplification of Normal DNA and Successful and Consistent Amplification of all Mutant DNA Templates.

As shown in the Figure there is a very strong block (delta CT between WT and variant over 20 cycles) and surprisingly consistent between mutations at all four positions (to within 2-3 cycles).

### EXAMPLE

Codons 12 & 13 of KRAS sequence (region of interest) can develop oncogenic mutations:

```
Codon number:
        9 10 11 12 13 14 15 16
DNA sequence:
5′ GTT GGA GCT GGT GGC GTA GGC AAG 3′
Amino Acid sequence:
      Val Gly Ala Gly Gly Val Gly Lys
```

These mutations comprise any of the three possible substitutions at one or more of first two bases in each codon (ie 4×3=12 possible substitutions).

| | |
|---|---|
| Codon 12: | Codon 13: |
| Gly 12 Asp GGT > GAT | Gly 13 Asp GGC > GAC |
| Gly 12 Ala GGT > GCT | Gly 13 Ser GGC > AGC |
| Gly 12 Val GGT > GTT | Gly 13 Arg GGC > CGC |
| Gly 12 Ser GGT > AGT | Gly 13 Val GGC > GTC |
| Gly 12 Arg GGT > CGT | Gly 13 Cys GGC > TGC |
| Gly 12 Cys GGT > TGT | Gly 13 Ala GGC > GCC |

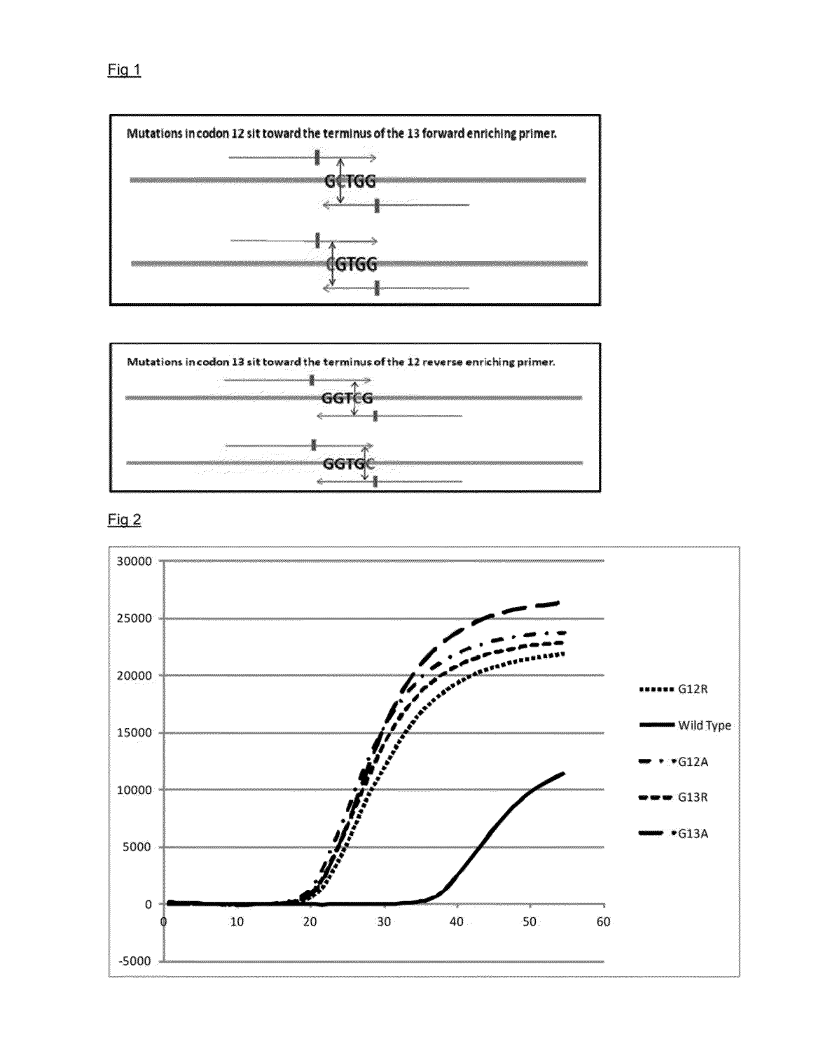

The enriching primers have a five base overlap encompassing the four bases of clinical significance within codons 12 & 13 (see FIG. 1).

All primers and probes used in the subsequent experiments were synthesized by Eurogentech. Real-time PCR and melting curve analysis were performed on BioRad IQ5. Primers were designed to amplify a target DNA sequence KRAS gene from plasmids comprising a normal KRAS gene fragment and four plasmids containing KRAS gene fragments with different mutations (harbouring either G12R, G12A, G13R or G13A). The sequence of this gene fragment comprises the sequence

atgactgaatataaacttgtggtagttggagctggtggcgtaggca
agagtgccttgacgatacagctaattcagaatcattttgtggacga
atatgatccaacaatagaggattcctacaggaagcaagtagtaatt
gatggagaaacctgtctcttggatattctcgacacagcaggt

The sequences of primers are:

KRAS13-enrich-f1
AAACTTGTGGTAGTTGGAGC1GGTGG
KRAS12-enrich-r1
GTCAAGGCACTCTTGCCT1CGCCACC
Point-KRAS-f1
GGTGGAGTATTTGATAGTGTATTAAC
Point-KRAS-r3
ACAAGATTTACCTCTATTGTTGGAT

The sequences of mutant templates are:

G12R
GTAGTTGGAGGTCGTGGCGTAGGCAAGAGT
G12A
GTAGTTGGAGCTGCTGGCGTAGGCAAGAGT
G13R
GTAGTTGGAGCTGGTCGCGTAGGCAAGAGT
G13A
GTAGTTGGAGCTGGTGCCGTAGGCAAGAGT

Wherein “1” is dspacer THF (Abasic site). All nucleic acid sequences are written 5′ to 3′ unless otherwise stated.

Primers were present at a final concentration of 300 nM for the amplifying primers and 600 nM for the enriching primers. Amplification was performed using the following ingredients and conditions: 10×PCR Buffer (stoffel fragment buffer from Applied Biosystems) 2.5 μl, 10 mM dNTPs 0.5 μl, each primer, if added, 0.5 μl, Stoffel fragment, AmpliTaq DNA polymerase (5 U/μl) 0.25 μl, plasmid DNA 0.5 μl (10⁵ molecules) and water to final volume of 25 μl. Reactions were carried out at 95° C. for 2 min; followed by 45 cycles of 95° C. for 9 s, 50° C. for 20 s, 70° C. for 1 s and 60° C. for 30 s. The primers added in reactions are as follows:

| Tube number | 1 | 2 | 3 | 4 | 5 |
|---|---|---|---|---|---|
| KRAS13-f | + | + | + | + | + |
| KRAS12-r | + | + | + | + | + |
| KRAS13-enrich-f | + | + | + | + | + |
| KRAS12-enrich-r | + | + | + | + | + |
| wild type DNA | + | − | − | − | − |
| G12R | − | + | − | − | − |
| G12A | − | − | + | − | − |
| G13R | − | − | − | + | − |
| G13A | − | − | − | − | + |

A typical amplification plot was obtained and is shown FIG. 2. Similar results were obtained on 2 different replications.

The example shows that one or more nucleotide variations, for example point mutations in a region of variants, can be enriched and detected by designing the enriching primer to have an appropriate 3′ DRBP which is complementary to the normal region such that the synthesis of the enriching primer extension product will block the extension of the upstream amplification primer Variant nucleotides at several positions can be enriched simultaneously in this manner by overlapping the forward and reverse enriching primers so that multiple positions are covered. Sub-terminal variant nucleotides even up to 5 nucleotides from the 3′ terminus can be enriched using enriching primers directed against the normal sequence. By overlapping the forward and reverse enriching primers several positions can be covered and therefore the further a variant nucleotide is from the 3′ terminus of one primer the closer it is to the 3′ terminus of the opposite enriching primer, allowing for consistent enrichment for variant nucleotides at a distance from the 3′ terminal nucleotides of either primer.

1. A method for enriching variant target nucleic acids from a population of reference nucleic acids,
wherein the nucleic acid includes a diagnostic region encompassing at least two potential variant nucleotides as compared to the reference sequence,
said method comprising:
(a) providing the population of nucleic acids in denatured form as complementary first and second single strands.
(b) providing forward and reverse enriching primers and forward and reverse amplification primers
wherein the forward and reverse enriching primers each include a 3′ diagnostic region binding portion (DREW) which is substantially complementary to the reference sequence of the entire diagnostic region of the first and second strands of the target nucleic acid respectively,
and wherein the nucleotide sequence of each of the forward and reverse amplification primer is substantially complementary to first and second strands of the target nucleic acid respectively at a region which is upstream of the forward and reverse enriching primer respectively,
(c) treating the denatured nucleic acid of (a) with the primers of (b) under hybridising conditions such as to form a mixture of duplexes
wherein each duplex comprises an enriching primer and an amplification primer annealed to each strand of the target nucleic acid,
wherein the DRBP of the enriching primer is annealed along the diagnostic region,
and wherein the amplification primer is annealed to the target nucleic acid such that the 3′ end of the amplification primer is upstream of the 5′ end of the enriching primer;
(d) maintaining the mixture of step (c) under extension conditions, which comprise appropriate nucleoside triphosphates and a nucleic acid polymerase to extend the annealed primers, if extendable to synthesize a population of nucleic acids including primer extension products,
wherein the extension of the enriching primer is inhibited by base-mismatching between the DRBP and the diagnostic region when the diagnostic region contains any one or more of the variant nucleotides, thereby allowing extension of the upstream amplification primer to pass through the diagnostic region containing any one or more of the variant nucleotides,
wherein the extension of the enriching primer is promoted where the DRBP is annealed to the reference diagnostic region, thereby synthesizing an enriching primer extension product which suppresses extension initiated from the upstream amplification primer,
thereby permitting preferential exponential amplification of the nucleic acid strands which include a diagnostic region in which any one or more of the variant nucleotides are present,
(e) repeating steps (c) and (d).

2. A method as claimed in claim 1 wherein the diagnostic region and the DRBP is 3, 4, 5, or 6 nucleotides length.

3. A method as claimed in claim 1 or claim 2 wherein the diagnostic region encompasses 2, 3, or 4 potential variant nucleotides,

4. A method as claimed in any one of claims 1 to 3 wherein the 3′ terminal nucleotide of the DRBP base pairs to one potential variant nucleotide position in the diagnostic region.

5. A method as claimed in any one of claims 1 to 4 wherein all the potential variant nucleotides are not consecutive.

6. A method as claimed in any one of claims 1 to 5 wherein the diagnostic region is an amino acid coding sequence and the potential variant nucleotides occur in consecutive codons.

7. A method as claimed in any one of claims 1 to 6 wherein each potential variant nucleotide can be substituted with 1, 2 or 3 non-wild type bases.

8. A method as claimed in any one of claims 1 to 7 wherein in step (b) the concentration of the enriching primers in greater than the concentration of the amplifying primers, optionally 2 fold greater or more.

9. A method as claimed in any one of claims 1 to 8 wherein the denaturing in step (a) is achieved by exposing the population of nucleic acid to a temperature of around 95 C for around 9 s.

10. A method as claimed in any one of claims 1 to 9 wherein the hybridising conditions of step (c) are a temperature of around 50 C for 20 s

11. A method as claimed in any one of claims 1 to 1 0 wherein the extension conditions of step (d) are a temperature of around 60° C. for 30 s.

12. A method as claimed in any one of claims 1 to 11 wherein the 3′ end of the amplification primer at least 20 bases upstream of the 5′ end of the enriching, primer in step (c).

13. A method as claimed in any one of claims 1 to 12 wherein one or both of the enriching primers comprise a moiety that renders the extension product of the enriching primer unsuitable for an exponential amplification.

14. A method as claimed in claim 13 wherein said moiety is a blocking moiety which is not suitable as a template for nucleic acid polymerase, wherein the replication of all or part of said enriching primer is blocked.

15. A method as claimed in claim 14 wherein said blocking moiety is a hydrocarbon arm , non-nucleotide linkage, peptide nucleic acid, nucleotide derivatives, abasic ribose or a dye.

16. A method as claimed in claim 14 or claim 15 wherein said blocking moiety is located less than 3, 6, or 18 nucleotides away from the 3′ terminus of the enriching primer.

17. A method as claimed in claim 13, wherein said moiety is a tail sequence of nucleotides or non-nucleic acid 5′ to the priming portion of the enriching primer, wherein the 5′ tail sequence is complementary or substantially complementary to an amplification primer binding site in the enriching primer extension product.

18. A method as claimed in any one of claims 1 to 12, wherein said appropriate nucleoside triphosphates comprise at lease one modified deoxynucleoside triphosphate, which renders a part or whole of an extended strand resistant to a nuclease cleavage, wherein said enriching primer comprises natural nucleotides and phosphodiester linkages, which render a part or whole of the enriching primer degradable by a nuclease activity.

19. A method as claimed in claim 18 wherein said nucleic acid polymerase comprises a 5° exonuclease activity, wherein said enriching primer is extended when it anneals to the diagnostic region containing the corresponding normal nucleotide on the target sequence, wherein a part or whole of the enriching primer is degraded by said 5′ exonuclease activity, whereas a part or whole of the extended portion of the extended enriching primer is resistant to cleavage.

20. A method as claimed in any one of claims 1 to 17 wherein step (e) further comprises treating the mixture under melting conditions to remove enriching primers from the diagnostic region where the diagnostic region contains any one or more of the variant nucleotides.

21. A method as claimed in any one of claims 1 to 17 wherein said enriching primer comprises modified nucleotides or linkages which render the whole or part of the enriching primer resistant to nuclease cleavage, wherein optionally the last 5 nucleotides or linkages at the 3′ end and/or 5′ end are modified such that the enriching primer is resistant to nuclease cleavage, and/or wherein the last nucleotide or linkage at the 3′ end and/or 5′ end are modified such that the enriching primer is resistant to nuclease cleavage.

22. A method as claimed in any one of claims 1 to 21 wherein said nucleic acid polymerase is a thermostable enzyme

23. A method as claimed in any one of claims 1 to 22 wherein steps (e) are performed as part of a PCR reaction.

24. A method as claimed in any one of claims 1 to 23 comprising detecting the enriched target nucleic acid.

25. A kit for performing a method of any one of claims 1 to 24.

26. A kit as claimed in claim 25 comprising:
(i) the forward and reverse enriching primers and forward and reverse amplification primers;
plus optionally one or more of:
(ii) target template DNA for use as a control;
(iii) one or more probes to facilitate detection of the enriched nucleic acid, wherein the probes and/or primers optionally comprises labels;
(iv) written instructions for performing the method;
(v) a nucleic acid polymerase which optionally comprises a 5′ exonuclease activity.

27. A method or kit as claimed in any one of claims 1 to 26 wherein the variant target nucleic acids are variants of the KRAS gene.


SEQUENCE LISTING (11 sequences)

SEQ ID NO: 1 - DNA, 24 bp, Homo sapiens
  CDS (1)..(24)
gttggagctggtggcgtaggcaag

SEQ ID NO: 2 - protein, 8 aa, Homo sapiens
VGAGGVGK

SEQ ID NO: 3 - DNA, 180 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggt

SEQ ID NO: 4 - DNA, 26 bp, Artificial sequence
  Synthetic sequence Primer KRAS13-enrich-f1
aaacttgtggtagttggagcnggtgg

SEQ ID NO: 5 - DNA, 26 bp, Artificial sequence
  Synthetic sequence Primer KRAS12-enrich-r1
gtcaaggcactcttgcctncgccacc

SEQ ID NO: 6 - DNA, 26 bp, Artificial sequence
  Synthetic sequence Primer Point-KRAS-f1
ggtggagtatttgatagtgtattaac

SEQ ID NO: 7 - DNA, 25 bp, Artificial sequence
  Synthetic sequence Primer Point-KRAS-r3
acaagatttacctctattgttggat

SEQ ID NO: 8 - DNA, 30 bp, Artificial sequence
  Synthetic sequence Mutant template G12R
gtagttggagctcgtggcgtaggcaagagt

SEQ ID NO: 9 - DNA, 30 bp, Artificial sequence
  Synthetic sequence Mutant template G12A
gtagttggagctgctggcgtaggcaagagt

SEQ ID NO: 10 - DNA, 30 bp, Artificial sequence
  Synthetic sequence Mutant template G13R
gtagttggagctggtcgcgtaggcaagagt

SEQ ID NO: 11 - DNA, 30 bp, Artificial sequence
  Synthetic sequence Mutant template G13A
gtagttggagctggtgccgtaggcaagagt